(19) United States
(12) Patent Application Publication
(10) Pub. No.: US 2024/0136135 A1
(43) Pub. Date: Apr. 25, 2024

(54) USE OF ONE OR MORE METRICS TO TRIGGER ELECTRIC PULSE INITIATION AND/OR TERMINATION DURING CONTROL OF ACTUATION OF A MEDICAMENT DELIVERY PUMP

(71) Applicant: INSULET CORPORATION, Acton, MA (US)

(72) Inventors: Eli WIEL, Boston, MA (US); Daniel BARRETT, Somerville, MA (US); Nicholas CONTE, Harvard, MA (US); Jerry WITT, Andover, MA (US)

(21) Appl. No.: 18/491,276

(22) Filed: Oct. 19, 2023

Related U.S. Application Data
(60) Provisional application No. 63/380,508, filed on Oct. 21, 2022.

(51) Int. Cl. H01H 71/14

(52) U.S. Cl. CPC H01H 71/145

(57) ABSTRACT

Exemplary embodiments may terminate application of an electric pulse to a shape memory alloy (SMA) element that causes actuation of a medicament pump based on resistance values unlike conventional approaches that rely on a mechanical mechanisms to trigger termination of the application of the electric pulse. The magnitude of the resistance values, the rate of change (RoC) of the resistance values, the temperature of the SMA element, the time that has passed since initial application of the electric pulse to the SMA element, or combinations thereof may be used to trigger the termination of the application of the electric pulse to the SMA element in exemplary embodiments. The monitoring of the resistance of an unactuated SMA element may be used to determine when to initiate and when to terminate application of an electrical pulse to the other SMA element.

### RELATED APPLICATIONS

This application claims the benefit of U.S. Provisional Patent Application No. 63/380,508, filed Oct. 21, 2022, the entire contents of which are incorporated herein by reference in its entirety.

### BACKGROUND

Certain conventional medicament delivery devices may employ shape memory alloy (SMA) elements. SMA elements transition between shapes as the temperatures of the elements change. SMA elements “remember” their original shapes when the temperature of the elements reaches a transition temperature. For example, an SMA wire may shorten to an original shorter shape when the SMA wire warms. This characteristic may be exploited within a device to actuate a component.

In one medicament delivery device provided by Insulet Corporation of Acton, Massachusetts, an SMA wire is used to drive the actuation of the medicament delivery pump of the device to deliver medicament. Specifically, an electric pulse of a predetermined duration is applied to the SMA wire in the device. The application of the electric pulse causes the SMA wire to heat and shorten. The shortening of the SMA wire causes a component to be moved that drives actuation of the medicament delivery pump. The termination of the application of the electric pulse to the SMA wire is caused by a mechanical termination mechanism that relies on overshoot of the component that drives actuation.

### SUMMARY

In accordance with an inventive facet, a medicament delivery device for delivering medicament to a user includes a medicament reservoir for storing the medicament and a medicament pump for pumping the medicament from the medicament reservoir for delivery to the user. The device also includes a power source and a shape memory alloy (SMA) element for causing actuation of the medicament pump to deliver the medicament. The device further includes a processor configured to initiate application of an electric pulse from the power source to the SMA element to cause the medicament pump to deliver the medicament from the medicament reservoir. The processor also is configured to determine resistance of the SMA element over time and based on the determined resistance of the SMA element over time, to determine whether the application of the electric pulse should be terminated. The processor additionally is configured to cause the application of the electric pulse to the SMA element to be terminated responsive to the determining that the application of the electric pulse should be terminated.

The processor may be configured to determine a rate of change (RoC) of the resistance and to compare the RoC to a threshold in the determining that the application of the electric pulse should be terminated. The processor may be configured to determine a magnitude of change of the resistance of the SMA element, to compare the determined magnitude of change of the resistance to a threshold, and to cause the electric pulse to terminate in part based on the comparing to the threshold. The processor may be configured to collect voltage readings and current readings for the SMA element over time, and the determining of the resistance of the SMA element over time may entail the processor calculating the resistance of the SMA over time from the collected voltage readings and current readings. The processor may cause the application of the electric pulse to the SMA element to be terminated by determining moving average values of subsets of the resistance values, determining at least one derivative or approximation of the derivative of the determined moving average values, and comparing the at least one derivative or the approximation of the derivative of the determined moving average values to a threshold as part of the determining that the application of the electric pulse should be terminated. The SMA element may be an SMA wire. The determining that the application of the electric pulse should be terminated may include determining a second derivative or an approximation of a second derivative of one of the resistance values or averages of successive ones of the resistance values and based on the second derivative or the approximation of the second derivative, determining that the application of the electric pulse should be terminated.

In accordance with another inventive facet, a medicament delivery device includes a medicament reservoir for storing medicament, a pump for pumping medicament from the medicament reservoir, a SMA element for actuating the pump, and a power source. The device also includes a processor configured to cause an electric pulse from the power source to be applied to the SMA element to cause actuation of the medicament pump to output medicament from the medicament reservoir, to monitor resistance values of the SMA element, and to terminate the application of the pulse to the SMA based on a metric reflective of the resistance values.

The medicament delivery device may be a wearable insulin pump, and the medicament may be insulin. The processor may be configured to perform filtering of the resistance values. The power source may be a battery and/or a capacitor. The SMA element may be coupled to a component that drives actuation of the medicament pump. The SMA element may shrink in length responsive to application of the electric pulse. The metric reflective of the resistance values may be a metric of a RoC over time concerning the resistance values or of averages of the resistance values. The metric of the RoC over time concerning the resistance values may be a derivative of the resistance values or averages of the resistance values. The metric of a RoC over time concerning the resistance values may be a second derivative of the resistance values or averages of the resistance values.

In accordance with an additional inventive aspect, a medicament delivery device includes a medicament reservoir for storing medicament, a pump for pumping medicament from the medicament reservoir, an SMA element for actuating the pump, a power source, and may further include a temperature sensor for sensing temperature of the SMA element, and a clock for outputting an indication of time. The device further includes a processor configured to cause an electric pulse from the power source to be initially applied to the SMA element to actuate the medicament pump to output medicament from the medicament reservoir. The processor may also be configured to monitor temperature values of the SMA element that were measured by the temperature sensor and to terminate the application of the pulse to the SMA element based on the temperature of the SMA element and time since the initial application of the electric pulse to the SMA element.

The SMA element may be one or more SMA wires. Multiple successive temperature values may be used to determine that a threshold has been exceeded before terminating the application of the pulse to the SMA element. The medicament delivery device may be an insulin delivery device.

In accordance with a further inventive aspect, a medicament delivery system may include a medicament reservoir for storing medicament and a pump for pumping medicament from the medicament reservoir. The system may include a first SMA element and a second SMA element for actuating the pump. The first and second SMA elements may be configured to be opposed and to be actuated in alternating fashion to drive the pump. The system may include a power source and a processor. The processor may be configured to monitor a resistance of the first SMA element. An electrical pulse from the power source may have been applied to the first SMA element more recently than the second SMA element. The processor may be further configured to identify when the resistance of the first SMA element reaches a threshold level and based on the identifying, to cause an electrical pulse to be applied to the second SMA element to activate the second SMA element in order to drive the pump to deliver medicament from the reservoir to a user.

The system may include one or more drive wheels coupled to the pump that are driven by the first SMA element and the second SMA element responsive to application of electrical pulses to the first and second SMA elements. The first SMA element and the second SMA element may be SMA wires. Application of the electrical pulse to the first SMA element may cause the first SMA element to transition from a current state to a more fully austenite state. The threshold level of resistance may be associated with a state where the second SMA element has not fully cooled to ambient temperature and is still partially in an austenite state. The monitoring of the resistance of the first SMA element may include measuring the voltage of the first SMA element and determining the resistance of the first SMA element from the measured voltage. The system may include a current sensor for measuring current at the first SMA element, and the measured current also may be used in determining the resistance of the first SMA element. The medicament may include at least one of insulin, a glucagon-like peptide (GLP)-1 receptor agonist, or a gastric inhibitory peptide (GIP), or a dual GIP-GLP receptor agonist.

In accordance with a still further inventive facet, a medicament delivery system may include a medicament reservoir for storing medicament and a pump for pumping medicament from the medicament reservoir. The system also may include a first SMA element and a second SMA element for actuating the pump. The first and second SMA elements may be configured to be opposed and actuated in alternating fashion to drive the pump. The system may include a power source and a processor. The processor may be configured to initiate application of an electrical pulse from the power source to the second SMA element to cause actuation of the second SMA element by transitioning from a current state to a more fully austenite state that results in actuation of the pump to deliver the medicament from the reservoir to the user. The processor also may be configured to monitor resistance of the second SMA element as the electrical pulse is applied to the second SMA element and based on the monitoring, to terminate application of the electrical pulse to the second SMA element.

The monitoring of the resistance of the second SMA element may include measuring a voltage of the second SMA element and a current of the second SMA element and determining the resistance from the measured voltage and the measured current. The application of the electrical pulse to the second SMA element may be terminated while a temperature of the second SMA element is higher than an ambient temperature. The first and second SMA elements may be SMA wires. The device may include a switch under control of the processor for the initiating of the application of the electrical pulse to the second SMA element. The switch also may be configurable to terminate the application of the electrical pulse to the second SMA element. The device may include an additional switch for controlling application of electricity to the first SMA element.

In accordance with yet another inventive facet, a method may include monitoring with a processor the resistances of a first SMA element and a second SMA element that are arranged in an opposing arrangement and are actuated in alternating fashion in a medical device. Per the method, based on the measured resistances, with the processor one of the following may be determined: that a crimp for one of the SMA elements has a problem, that one of the SMA elements overheated during application of an electrical stimulus, or that there is a problem with a ground for an element of the medical device.

The medical device may be a medicament delivery device. The SMA elements may act as actuators for a pump to cause delivery of medicament from the pump. The SMA elements may be SMA wires. The opposing arrangement may be configured to cause a selected one of the SMA elements to stretch the other SMA element as the other SMA element cools. The method may include obtaining voltage and current values to calculate the resistances of the SMA elements.

### BRIEF DESCRIPTION OF THE DRAWINGS

FIG. 1 depicts an illustrative medicament delivery system of exemplary embodiments.

FIG. 2 depicts internal components of an illustrative medicament delivery device of exemplary embodiments.

FIG. 3 depicts a flowchart of illustrative steps that may be performed in exemplary embodiments to terminate the application of an electric pulse to an SMA element based on resistance values for the SMA element.

FIG. 4 depicts an illustrative plot of resistance of an SMA element of a medicament delivery device when an electric pulse is applied to the SMA element.

FIG. 5 depicts illustrative plots of resistance and rate of change (RoC) of resistance of an SMA element of a medicament delivery device when an electric pulse is applied to the SMA element.

FIG. 6 depicts a flowchart of illustrative steps that may be performed in exemplary embodiments to terminate application of an electric pulse to an SMA element based of RoC concerning resistance values.

FIG. 7 depicts a flowchart of illustrative steps that may be performed in exemplary embodiments to calculate the RoC from differences in consecutive resistance values.

FIG. 8 depicts a flowchart of illustrative steps that may be performed in exemplary embodiments to calculate the RoC as the first derivative of resistance values relative to time.

FIG. 9 depicts a flowchart of illustrative steps that may be performed in exemplary embodiments to calculate the RoC as the second derivative of resistance values relative to time.

FIG. 10 depicts a flowchart of illustrative steps that may be performed in exemplary embodiments to terminate application of an electric pulse to the SMA element based on RoC concerning resistance values and a magnitude of change in the resistance values.

FIG. 11 depicts a flowchart of illustrative steps that may be performed in exemplary embodiments to terminate application of an electric pulse to the SMA element based on elapsed time.

FIG. 12 depicts a flowchart of illustrative steps that may be performed in exemplary embodiments to terminate application of an electric pulse to the SMA element based on multiple factors.

FIG. 13 depicts a flowchart of illustrative steps that may be performed in exemplary embodiments to terminate application of an electric pulse to the SMA element based on temperature of the SMA element.

FIG. 14 depicts a flowchart of illustrative steps that may be performed in exemplary embodiments to terminate application of an electric pulse to the SMA element based on temperature of the SMA element and elapsed time.

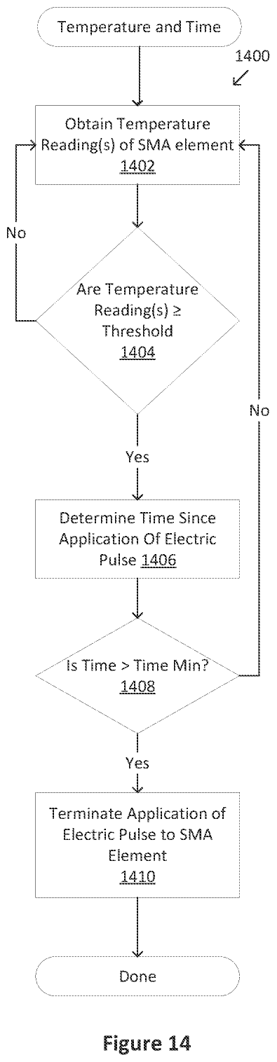

FIG. 15 depicts a flowchart of illustrative steps that may be performed in exemplary embodiments to determine the state of an SMA element from the resistance of the SMA element.

FIG. 16 depicts examples of state information for an SMA element that may be determined from resistance measurements in exemplary embodiments.

FIG. 17 depicts an illustrative chart of pulse timing for two opposing SMA elements is exemplary embodiments.

FIG. 18 depicts a flowchart of illustrative steps that may be performed in exemplary embodiments to trigger application of a pulse to an SMA element based on resistance values for an unactuated SMA element.

FIG. 19 depicts a flowchart of illustrative steps that may be performed in exemplary embodiments to trigger termination of application of a pulse to an SMA element based on resistance values for an unactuated SMA element.

FIG. 20 depicts an illustrative plot resistance of an SMA element overlayed on the pulse chart diagram of FIG. 17.

FIG. 21 depicts an illustrative electric circuit for monitoring resistance of an SMA element in exemplary embodiments.

FIG. 22A depicts a flowchart of illustrative steps of a first option that may be performed in exemplary embodiments to address a voltage offset issue.

FIG. 22B depicts a flowchart of illustrative steps of a second option that may be performed in exemplary embodiments to address a voltage offset issue.

FIG. 23 depicts an alternative electrical circuit for monitoring the resistance of an SMA element in exemplary embodiments.

FIG. 24 depicts another alternative electrical circuit for monitoring the resistance of an SMA element in exemplary embodiments.

FIG. 25 depicts a flowchart of illustrative steps of a first option that may be performed in exemplary embodiments to determine whether an SMA element overheated.

FIG. 26 depicts a flowchart of illustrative steps of a first option that may be performed in exemplary embodiments to identify crimp or hook ground issues based on monitored resistance values for SMA elements.

### DETAILED DESCRIPTION

Exemplary embodiments may terminate application of an electric pulse to an SMA element that causes actuation of a medicament pump based on resistance values unlike conventional approaches that rely on mechanical mechanisms (including mechanical-electrical switch mechanisms) to trigger termination of the application of the electric pulse. The magnitude of the resistance values, the rate of change (RoC) concerning the resistance values, the temperature of the SMA element, the time that has passed since initial application of the electric pulse to the SMA element, or combinations thereof may be used to trigger the termination of the application of the electric pulse to the SMA element in exemplary embodiments, as will be described below. It should be appreciated that RoC concerning the resistance of the SMA element may be captured by the first or second derivative of resistance values relative to time as described below. The RoC concerning the resistance values may be a more robust and reliable metric for accurately triggering termination of application of the electric pulse at a desired time than merely relying on resistance value magnitudes.

More accurate timing (i.e., earlier) termination of application of the electric pulse to the SMA element in the exemplary embodiments results in less energy use during operation of the medicament delivery device. This may be especially beneficial when the medicament delivery device is powered by a finite power source, like batteries and/or a charged capacitor, where the lower energy requirements can extend the time period before the power source needs to be recharged or replaced. The earlier termination may also result in less mechanical fatigue on the SMA element due to the shortened excitation time of the wire resulting from the earlier termination of application of electric pulses relative to the conventional termination approach.

In some exemplary embodiments, the resistance of the unactuated SMA element (i.e., the SMA element that was most recently actuated but is not currently actuated) may be monitored. This monitoring may be used to determine when to initiate application of an electrical pulse to the other SMA element. Further, the monitoring of the resistance of the unactuated SMA element may be used to decide when to terminate application of the electrical pulse to the other SMA element. The initiation of the pulse may occur before the unactuated SMA element is fully cooled, and the termination of the electrical pulse may be chosen to be as soon as possible to save energy and extend the lifetime of the SMA elements.

The monitoring of the resistances of the SMA elements may also identify issues within the device. For instance, the monitoring of the resistances may be used to identify overheating of an SMA element or problems with a crimp or a ground of a component. More generally, the monitoring of the resistances of the SMA elements may provide useful information regarding the states of the SMA elements.

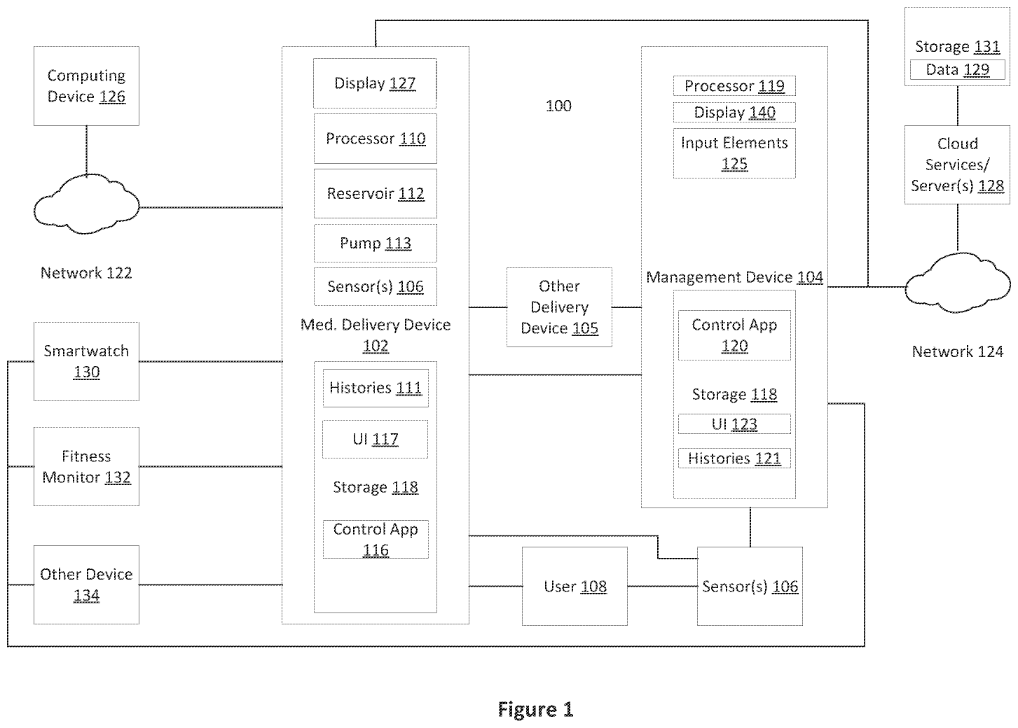

FIG. 1 depicts a block diagram of an illustrative medicament delivery system 100 that is suitable for delivering a medicament to a user 108 in accordance with the exemplary embodiments. The medicament delivery system 100 includes a medicament delivery device 102. The medicament delivery device 102 may be a wearable device that is worn on the body of the user 108 or carried by the user. The medicament delivery device 102 may be directly coupled to a user (e.g., directly attached to a body part and/or skin of the user 108 via an adhesive or the like) with no tubes and an infusion location directly under the medicament delivery device 102, or carried by the user (e.g., on a belt or in a pocket) with the medicament delivery device 102 connected to an infusion site where the medicament is injected using a needle and/or cannula. A surface of the medicament delivery device 102 may include an adhesive to facilitate attachment to the user 108.

The medicament delivery device 102 may include a processor 110. The processor 110 may be, for example, a microprocessor, a logic circuit, a field programmable gate array (FPGA), an application specific integrated circuit (ASIC) or a microcontroller. The processor 110 may maintain a date and time as well as other functions (e.g., calculations or the like). The processor 110 may be operable to execute a control application 116 encoded in computer programming instructions stored in the storage 114 that enables the processor 110 to direct operation of the medicament delivery device 102. The control application 116 may be a single program, multiple programs, modules, libraries or the like. The processor 110 also may execute computer programming instructions stored in the storage 114 for a user interface (UI) 117 that may include one or more display screens shown on display 127. The display 127 may display information to the user 108 and, in some instances, may receive input from the user 108, such as when the display 127 is a touchscreen.

The control application 116 may control delivery of a medicament to the user 108 per a control approach like that described herein. In exemplary embodiments, the control application 116 may control the termination of the electric pulse to an SMA element as described below. The storage 114 may hold histories 111 for the device and/or user, such as a history of resistance values, resistance RoC values, or a history of basal deliveries, a history of bolus deliveries, and/or other histories, such as a meal event history, exercise event history, glucose level history, and/or the like. In addition, the processor 110 may be operable to receive data or information. The storage 114 may include both primary memory and secondary memory. The storage 114 may include random access memory (RAM), read only memory (ROM), optical storage, magnetic storage, removable storage media, solid state storage or the like.

The medicament delivery device 102 may include a tray or cradle and/or one or more housings for housing its various components including a pump 113, a power source (not shown), and a reservoir 112 for storing a medicament for delivery to the user 108. A fluid path to the user 108 may be provided, and the medicament delivery device 102 may expel the medicament from the reservoir 112 to deliver the medicament to the user 108 using the pump 113 via the fluid path. The fluid path may, for example, include tubing coupling the medicament delivery device 102 to the user 108 (e.g., tubing coupling a cannula to the reservoir 112), and may include a conduit to a separate infusion site. The medicament delivery device 102 may have operational cycles, such as every 5 minutes, in which basal doses of medicament are calculated and delivered as needed. These steps are repeated for each cycle.

There may be one or more elements for enabling communications links with one or more devices physically separated from the medicament delivery device 102 including, for example, a management device 104 of the user and/or a caregiver of the user, sensor(s) 106, a smartwatch 130, a fitness monitor 132 and/or another variety of device 134. The communication links may include any wired or wireless communication links operating according to any known communications protocol or standard, such as Bluetooth®, Wi-Fi, a near-field communication standard, a cellular standard, or any other wireless protocol.

The medicament delivery device 102 may interface with a network 122 via a wired or wireless communications link. The network 122 may include a local area network (LAN), a wide area network (WAN) or a combination therein. A computing device 126 may be interfaced with the network 122, and the computing device may communicate with the medicament delivery device 102.

The medicament delivery system 100 may include one or more sensor(s) 106 for sensing the levels of one or more analytes. The sensor(s) 106 may be coupled to the user 108 by, for example, adhesive or the like and may provide information or data on one or more medical conditions and/or physical attributes of the user 108. The sensor(s) 106 may be physically separate from the medicament delivery device 102 or may be an integrated component thereof. The sensor(s) 106 may include, for example, glucose monitors, such as continuous glucose monitors (CGM's) and/or non-invasive glucose monitors. The sensor(s) 106 may include ketone sensors, analyte sensors, heart rate monitors, breathing rate monitors, motion sensors, temperature sensors, perspiration sensors, blood pressure sensors, alcohol sensors, or the like. Some sensors 106 may also detect characteristics of components of the medicament delivery device 102. For instance, the sensors 106 in the medicament delivery device may include voltage sensors, current sensors, temperature sensors and the like.

The medicament delivery system 100 may or may not also include a management device 104. In some embodiments, no management device is needed as the medicament delivery device 102 may manage itself. The management device 104 may be a special purpose device, such as a dedicated personal diabetes manager (PDM) device. The management device 104 may be a programmed general-purpose device, such as any portable electronic device including, for example, a dedicated controller, such as a processor, a micro-controller, or the like. The management device 104 may be used to program or adjust operation of the medicament delivery device 102 and/or the sensor(s) 106. The management device 104 may be any portable electronic device including, for example, a dedicated device, a smartphone, a smartwatch, or a tablet. In the depicted example, the management device 104 may include a processor 119 and a storage 118. The processor 119 may execute processes to manage a user's glucose levels and to control the delivery of the medicament to the user 108. The medicament delivery device 102 may provide data from the sensors 106 and other data to the management device 104. The data may be stored in the storage 118. The processor 119 may also be operable to execute programming code stored in the storage 118. For example, the storage 118 may be operable to store one or more control applications 120 for execution by the processor 119. The control application 120 may be responsible for controlling the medicament delivery device 102, such as by controlling the automated insulin delivery (AID) of insulin to the user 108. In some exemplary embodiments, the control application 120 provides the adaptability described herein. The storage 118 may store the control application 120, histories 121 like those described above for the medicament delivery device 102, and other data and/or programs.

A display 140, such as a touchscreen, may be provided for displaying information. The display 140 may display user interface (UI) 123. The display 140 also may be used to receive input, such as when it is a touchscreen. The management device 104 may further include input elements 125, such as a keyboard, button, knobs, or the like, for receiving input form the user 108.

The management device 104 may interface with a network 124, such as a LAN or WAN or combination of such networks, via wired or wireless communication links. The management device 104 may communicate over network 124 with one or more servers or cloud services 128. Data, such as sensor values, may be sent, in some embodiments, for storage and processing from the medicament delivery device 102 directly to the cloud services/server(s) 128 or instead from the management device 104 to the cloud services/server(s) 128.

Other devices, like smartwatch 130, fitness monitor 132 and device 134 may be part of the medicament delivery system 100. These devices 130, 132 and 134 may communicate with the medicament delivery device 102 and/or management device 104 to receive information and/or issue commands to the medicament delivery device 102. These devices 130, 132 and 134 may execute computer programming instructions to perform some of the control functions otherwise performed by processor 110 or processor 119, such as via control applications 116 and 120. These devices 130, 132 and 134 may include displays for displaying information. The displays may show a user interface for providing input by the user, such as to request a change or pause in dosage, or to request, initiate, or confirm delivery of a bolus of a medicament, or for displaying output, such as a change in dosage (e.g., of a basal delivery amount) as determined by processor 110 or management device 104. These devices 130, 132 and 134 may also have wireless communication connections with the sensor 106 to directly receive analyte measurement data. Another delivery device 105, such as a medicament delivery pen, may be accounted for or may be provided for also delivering medicament to the user 108.

A wide variety of medicaments may be delivered by the medicament delivery device 102 and delivery device 105. The medicament may be insulin for treating diabetes. The medicament may be glucagon for raising a user's glucose level. The medicament may also be a glucagon-like peptide (GLP)-1 receptor agonists for lowering glucose or slowing gastric emptying, thereby delaying spikes in glucose after a meal. Alternatively, the medicament delivered by the medicament delivery device 102 may be one of a pain relief agent, a chemotherapy agent, an antibiotic, a blood thinning agent, a hormone, a blood pressure lowering agent, an antidepressant, an antipsychotic, a statin, an anticoagulant, an anticonvulsant, an antihistamine, an anti-inflammatory, a steroid, an immunosuppressive agent, an antianxiety agent, an antiviral agent, a nutritional supplement or a vitamin. The medicament may be a coformulation of two or more of those medicaments listed above.

The functionality described herein for the exemplary embodiments may be under the control of or performed by the control application 116 of the medicament delivery device 102 or the control application 120 of the management device 104. In some embodiments, the functionality wholly or partially may be under the control of or performed by the cloud services/servers 128, the computing device 126 or by the other enumerated devices, including smartwatch 130, fitness monitor 132 or another wearable device 13

In the closed loop mode, the control application 116, 120 determines the medicament delivery amount for the user 108 on an ongoing basis based on a feedback loop. For an insulin delivery device, the aim of the closed loop mode is to have the user's glucose level at a target glucose level or within a target glucose range.

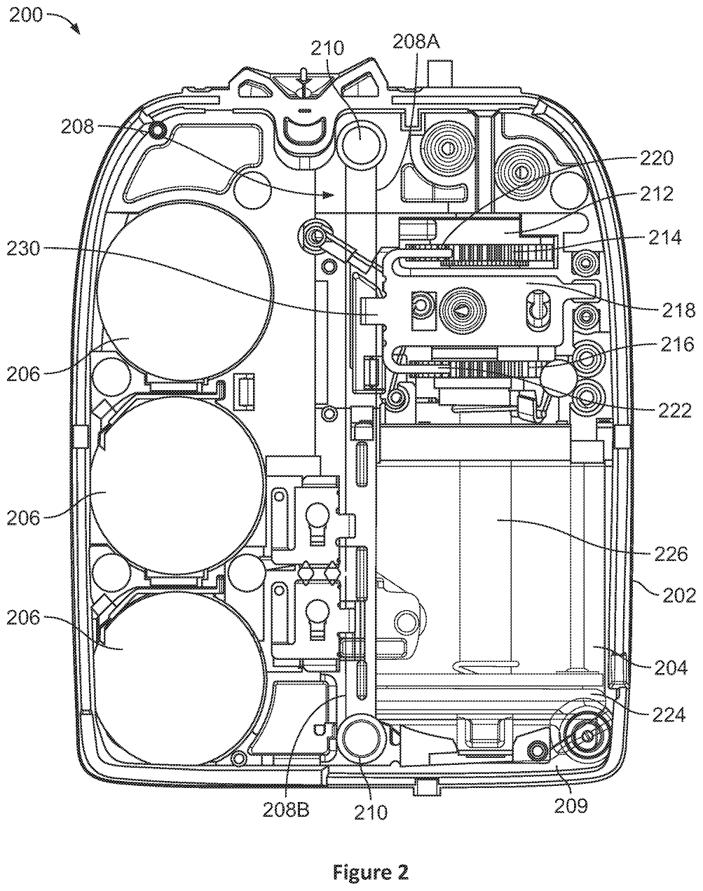

FIG. 2 depicts exemplary components found inside the housing 202 of an exemplary medicament delivery device 200. The components may include a reservoir 204 in which medicament is stored for delivery to the user 108, batteries 206 to serve as a power source for the medicament delivery device 200, SMA wire 208 (formed of a wire 208A and a second wire 208B that are collectively referred to as 208) for causing actuation of a medicament pump 209. The SMA wires 208 may be wrapped around SMA pulleys 210. A drive wheel 212 is coupled to the medicament pump 209 and includes one or more toothed wheels 214 and 216. A pivotable drive engaging member 218 has one or more arms 220 and 222 for engaging the toothed wheel 214 and 216, respectively. The SMA wire 208 is coupled to the pivotable drive engaging member 218 by integral connector 230 to cause the pivotable drive engaging member 218 to pivot back and forth and drive the drive wheel 212 as described below. The medicament pump 209 pumps the medicament from the reservoir 204. The medicament pump 209 includes a plunger 224 that actuates to expel medicament from the reservoir 204. The drive wheel 212 may be connected to tube nut 226. The tube nut 226 may be positioned on a leadscrew (not shown) on the plunger 224, and movement of the drive wheel 212 may cause the leadscrew to rotate, which in turn causes linear displacement of the plunger 224.

To actuate the exemplary medicament pump 209, an electric pulse is applied to SMA wire 208A to energize the SMA wire 208A. When charged, the first portion of the SMA wire 208 contracts and pulls the pivotable drive engaging member 218 in a first direction. When the pivotable drive engaging member 218 pivots in the first direction, the arm 220 engages a tooth on the toothed wheel 214 causing the drive wheel 212 to rotate one increment. The pivotable drive engaging member 262 pivots in the first direction. Generally speaking, one of the arms 220 and 222 is alternatively engaged by the toothed wheels 214 and 216 of the drive wheel 212. The engaged arm 220 and 222, therefore prevent reverse rotation of the drive eliminating the need for a separate pawl element.

To initiate another pulse, the control circuitry applies current to SMA wire 208B. When charged, the SMA wire 208B contracts and pulls the pivotable drive engaging member 218 in a second direction that is the opposite of the first direction. When the pivotable drive engaging member 218 pivots in the second direction, the arm 222 engages a tooth on the toothed wheel 216 causing the drive wheel 212 to rotate one increment. The pivotable drive engaging member 218 pivots in the second direction.

Each incremental rotation of the drive wheel 212 advances the plunger in the reservoir 204 to cause a discrete amount of fluid to be dispensed. The discrete amount of fluid to be dispensed is a function of the lead screw pitch of the (i.e., threads/inch), toothed wheel tooth size and the diameter of the fluid reservoir. In a preferred embodiment, for delivering U100 insulin for treatment of Type I diabetes, the discrete amount of fluid to be dispensed is between about 0.025 ul and about 0.05 ul. The control circuitry alternates energizing the SMA wires s 208A and 208B until a desired amount of fluid has been dispensed.

The depiction of the exemplary medicament delivery device 200 in FIG. 2 shows the mechanical overshoot components in place. Nevertheless, those components may be removed or may remain in the exemplary embodiments.

The optimal time for terminating the application of the electric pulse to the SMA wires 208A and 208B corresponds to the point in time where the active arm 220 or 222 of the pivotable drive engaging member 218 falls off the corresponding toothed wheel 214 or 216 of the drive wheel 212 of the medicament pump 209. The exemplary embodiments may determine and use a termination time that is closer to the optimal termination time than a conventional approach that relies upon a mechanical overshoot mechanism.

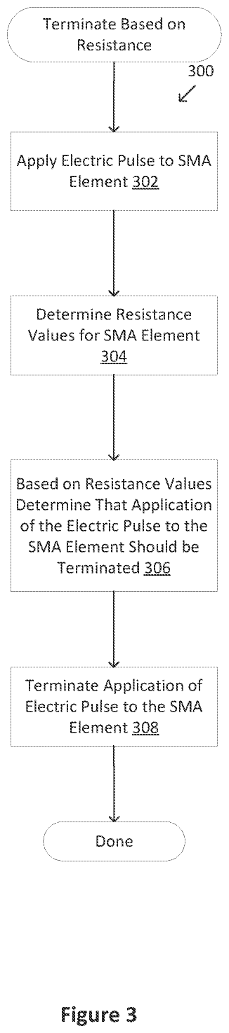

As was mentioned above, in exemplary embodiments a resistance metric (e.g., resistance values, RoC of resistance values, etc.) may be used in triggering the termination of an electric pulse that drives actuation of a medicament pump 113 in the medicament delivery device 102. FIG. 3 depicts a flowchart of illustrative steps that may be performed in exemplary embodiments to use resistance values in triggering the termination of the electric pulse. The termination in the electric pulse to the SMA element (like SMA wires 208) causes the medicament pump 113 to no longer actuate and thus to cease delivery of medicament to the user 108. At 302, the electric pulse is applied to the SMA element, such as the SMA wire 208A or 208B, to cause actuation of the medicament pump 113 resulting in delivery of medicament to the user 108 from the medicament delivery device 102. At 304, the resistance values of the SMA element are monitored to determine the resistance of the SMA element over time. Based on the determined resistance values, at 306, a determination is made that the electric pulse should be terminated so that the electric pulse is no longer applied to the SMA element. At 308, based on the determination, application of the electric pulse to the SMA element is terminated.

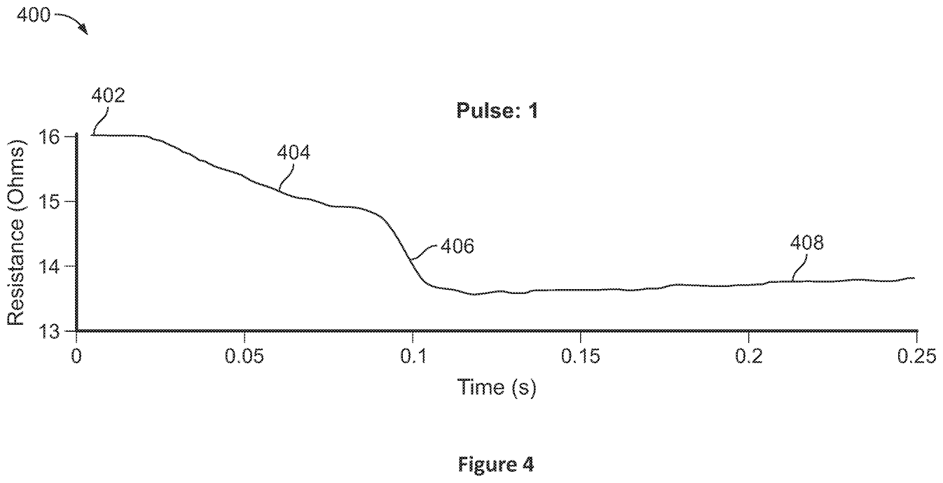

FIG. 4 depicts an illustrative plot 400 of resistance expressed in ohms of the SMA element over time during application of an electric pulse of 0.25 seconds. In this instance the SMA element is one of the SMA wires 208A or 208B. When the electric pulse is initially applied at 402, the resistance is at its highest level of approximately 16 ohms. As the electricity flows through the SMA wire 208 responsive to the electric pulse, the SMA wire 208A or 208B heats, and the resistance of the SMA wire 208A or 208B slowly begins to drop. Hence, at 404, the resistance has dropped relative to 402. As the SMA wire 208A or 208B continues to heat, the SMA wire 208A or 208B approaches and then undergoes a state transition from a martensite state to an austenite state. The SMA wire 208A or 208B shortens as the transition progresses and the resistance decreases more quickly at 406 in the plot 400 as the transition temperature where the state transition occurs is reached. Due to the arrangement of the actuation components of the medicament pump (e.g., toothed wheels 214, 216, arms 220, 222, pivotable drive engaging member 218, and the SMA wire(s) connected thereto), it is during this time that the actuation of the toothed wheel 214 and 216 by the arm 220 or 222 is completed responsive to movement of the SMA wire 208A or 208B. Once the transition is complete, the resistance stays largely flat since the SMA wire 208A or 208B is no longer shortening as indicated by the flat region at 408 in the plot 400.

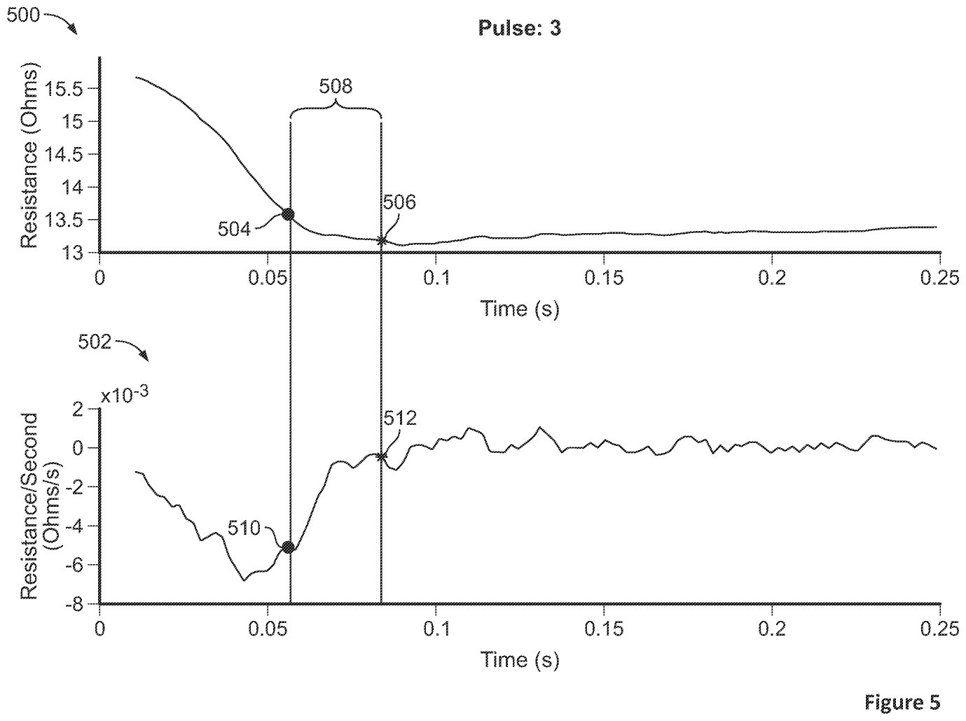

FIG. 5 depicts a plot 500 that illustrates the earlier or more accurate termination on the plot of resistance values versus time. This plot 500 is derived based on resistance values and termination points triggered based on use of a conventional mechanical overshoot termination mechanism on the one hand and, on the other hand, use of the termination approach based on resistance values on the same device as described herein. At 504, the application of the electric pulse to the SMA wire is terminated based on resistance values, such as in the exemplary embodiments. As a safety factor, there is some latency between the detection and the termination so that the termination is not at the true optimal termination point. Later, at 506, application of the electric pulse to the SMA wire is terminated by the mechanical overshoot mechanism employed in the conventional system.

FIG. 5 also shows plot 502, which depicts the RoC in resistance over time. Point 510 corresponds to the time of termination based on resistance values and point 512 corresponds to the time of termination based on the conventional mechanical overshoot termination mechanism.

The earlier termination of application of the electric pulse to the SMA element in the exemplary embodiments results in less energy use during operation of the medicament delivery device 102. This may be especially beneficial when the medicament delivery device 102 is powered by batteries, where the lower energy requirements can extend the battery life. The earlier termination may also result in less mechanical fatigue on the SMA element due to the shortened excitation time of the wire resulting from the earlier termination of application of electric pulses relative to the conventional termination approach.

The resistance values of the SMA element, such as the SMA wires 208A or 208B, may be obtained from a resistance measuring component. Alternatively, the resistance values may be obtained by determining the voltage applied to the SMA element and measuring the current flowing through the SMA element. Ohm's law states that I=V/R, where I is current, V is voltage, and R is resistance. Thus, resistance can be calculated as R=VA. Hence, once the voltage and current are obtained, the resistance for the SMA element may be determined.

A first approach that may be adopted to trigger termination of the electric pulse to the SMA element is to examine a RoC concerning the resistance of the SMA element (e.g., the SMA wire). In this context, the RoC may refer to the difference in resistance between successively sampled resistance values or averages of resistance values, such as determined from a first derivative or an approximation of a first derivative relative to time of resistance values or averages of resistance values, or deceleration of change in resistance values, such as determined from a second derivative of resistance values. The resistance values may be sampled at predetermined sample times. As detailed below, the RoC may, in some exemplary embodiments, be one of multiple factors that are reviewed in deciding whether to terminate application of the electric pulse to the SMA element.

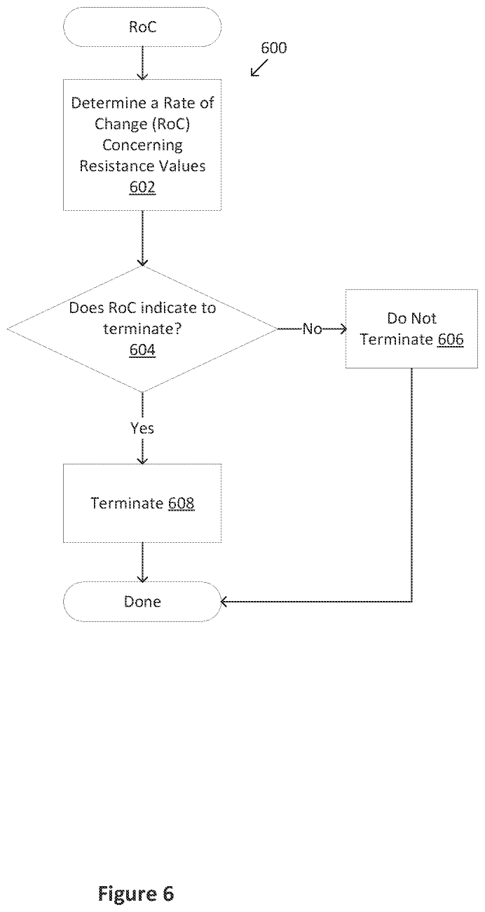

FIG. 6 depicts a flowchart 600 of illustrative steps that may be performed in exemplary embodiments in using the RoC concerning resistance values to trigger termination of the application of the electric pulse to the SMA element that causes actuation of the medicament pump 113. At 602, the RoC concerning the resistance values may be determined. A number of illustrative approaches for determining the RoC are detailed below. At 604, the determined RoC is compared to a threshold to determine if should the application of the electric pulse should be terminated. If not, at 606, the application of the electric pulse is not terminated. If so, at 608, the application of the electric pulse to the SMA element is terminated.

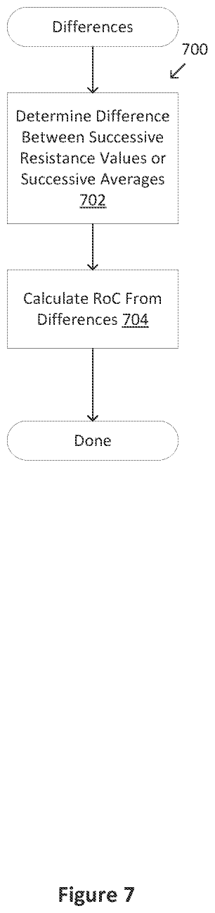

FIG. 7 depicts a flowchart 700 of illustrative steps that may be performed in exemplary embodiments to determine the RoC (see 602) in accordance with a first approach. In this first approach, at 702, the differences between resistance values at successive times or between averages, such as rolling averages, at successive times is determined. At 704, the RoC is calculated from the difference, such as being set equal to the difference. Where the RoC is determined by determining the difference between resistance values or the difference between averages of resistance values at successive times (such as rolling averages of several successive resistance values), the comparison at 604 may be whether the RoC is below a threshold value (which may be determined empirically). This is because the resistance of the SMA element does not continue to drop and may actually rise slightly after actuation responsive to the electric pulse is complete as shown in FIG. 4.

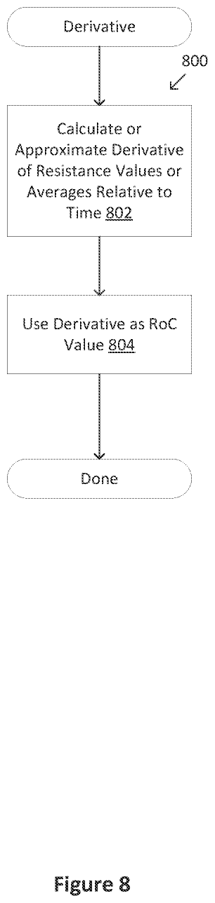

Another approach to determining the RoC value is to determine a first derivative or an approximation of the first derivative of the resistance values as the RoC. FIG. 8 depicts a flowchart 800 of illustrative steps that may be performed in exemplary embodiments. At 802, the first derivative of resistance relative to time (i.e., dR/dt) is calculated or an approximation of the first derivative is calculated. In some embodiments, a curve may be constructed from the obtained resistance values and interpolation may be used between values to complete the curve. At 804, the calculated or approximated first derivative may be used as the RoC value.

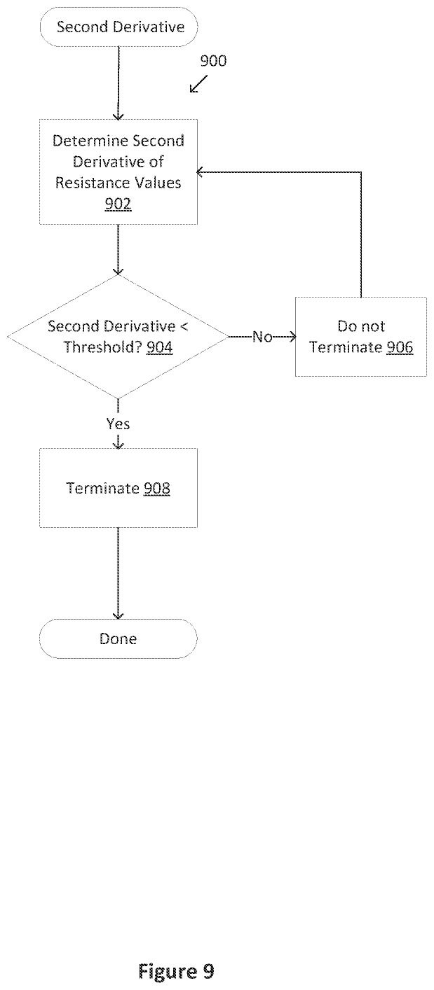

An additional approach to determining the RoC value is to use a second derivative of the resistance values as the RoC. The second derivative determines the RoC of the RoC of the resistance values. FIG. 9 depicts a flowchart 900 of illustrative steps that may be performed in exemplary embodiments to use the second derivative. At 902, a second derivative of the resistance values is determined. At 904, a check of whether the second derivative is less than a threshold is performed. At the inflection point where the state transition is complete and the resistance has stopped decelerating, the acceleration or deceleration of the change in resistance values should zero or near zero, since the RoC of the resistance values does not change a great deal at the inflection point. If not, at 906, the application of the electric pulse to the SMA element is not terminated and recalculated at the next sample time. If so, at 908, application of the electric pulse to the SMA element is terminated.

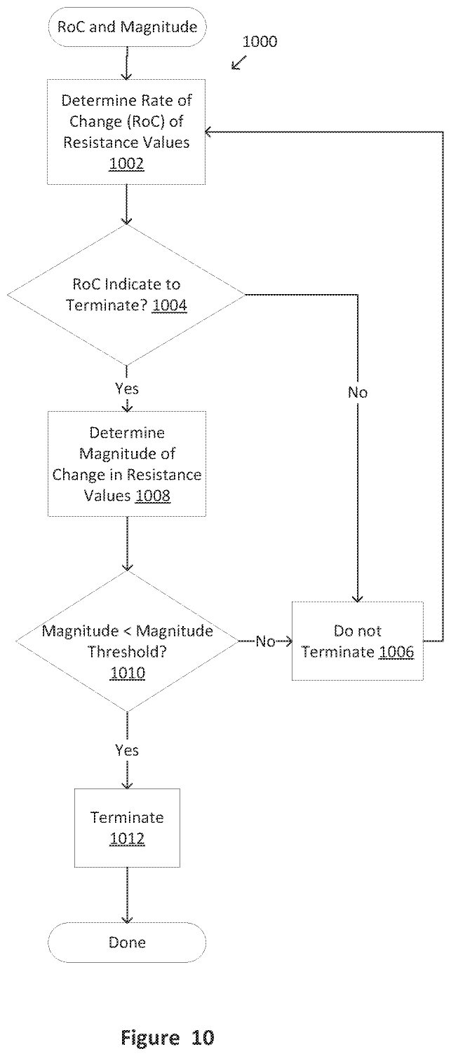

In some exemplary embodiments, the RoC concerning the resistance values and the magnitude of change in resistance since a last sampling time are used in conjunction to determine whether to trigger termination of application of the electric pulse to the SMA element. Looking at the magnitude of change in the resistance values helps avoid triggering termination early due to noise or other factors. FIG. 10 depicts a flowchart 1000 of illustrative steps that may be performed in exemplary embodiments to trigger termination of application of the electric pulse based on RoC of the resistance values and the percentage of change in the resistance values. At 1002, the RoC of the resistance values is determined, such as described above. If the RoC of the resistance values does not indicate that the application of the electric pulse should be terminated at 1004, then the application of the electric pulse is not terminated at 1006 and the process repeats at 1002. If the RoC of the resistance values indicates that there should be termination at 1004, then additional steps are performed. At 1008, the percentage of the change in resistance between samples is determined, and at 1010, the percentage is compared to a threshold. If the percentage is not less than the threshold, the application of the electric pulse is not terminated at 1006 and the process repeats at 1002. If the percentage of the change in resistance values is less than the threshold, at 1012, the application of the electric pulse to the SMA element is terminated. It should be appreciated that measures of magnitude of change other than percentages of change may be used in some embodiments.

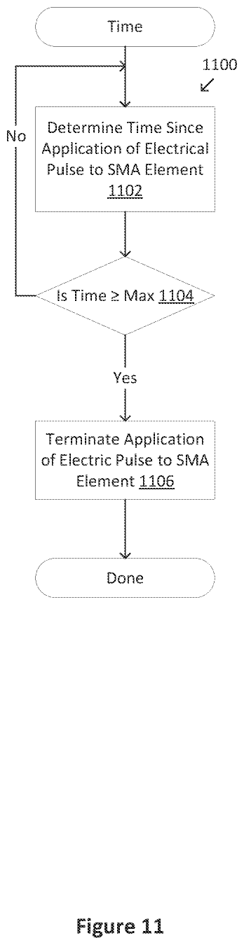

As a stop gap measure or as an alternate approach, the time that has elapsed since application of the electric pulse to the SMA element may be used as a metric to terminate application of the electric pulse to the SMA element in some exemplary embodiments. FIG. 11 depicts a flowchart 1100 of illustrative steps that may be performed in exemplary embodiments to use time to trigger termination of application of the electric pulse to the SMA element. At 1102, the time since the initiation of the application of the electric pulse to the SMA element is determined using a timer or based on a clock that is accessible to or incorporated in the processor 110. At 1104, a check is made whether the time has exceeded a maximum threshold. The maximum threshold may be a time close to the optimal termination time or may be fail safe time to terminate application of the pulse that otherwise would continue longer than desired. If the maximum time is exceeded, at 1106, application of the electric pulse to the SMA element is terminated. If not, the process repeats beginning at 1102.

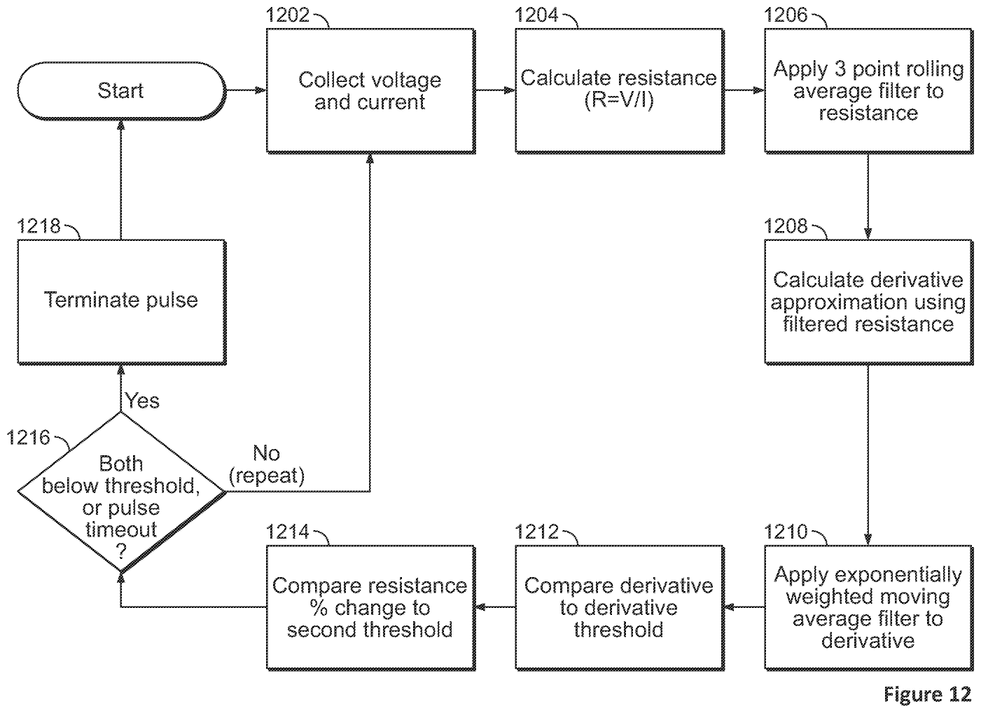

As was mentioned above, different combinations of metrics and possibly other values may be used to trigger termination of application of the electric pulse to the SMA element in exemplary embodiments. FIG. 12 depicts a flowchart 1200 of illustrative steps that may be performed in exemplary embodiments where the derivative of resistance values at a point in time and the resistance percentage change are used in conjunction to trigger termination of application of the electric pulse to the SMA element. At 1202, the voltage drop across the SMA element and the current are collected. These values are used at 1204 to calculate the resistance value at 1204. An exemplary three point rolling average filter may be applied at 1206 to output a three point moving average of the latest resistance value and the previous two resistance values. The first derivative is approximated based on the three point moving average at 1208. An exponentially weighted moving average filter is applied to the derivative at 1210 to determine an exponentially weighted moving average of the derivative values. The resulting derivative average is compared to a first threshold at 1212. The percentage change in resistance is determined and compared to a second threshold at 1214. If the derivative and the percentage change of the resistance are below the thresholds to which they are compared as checked at 1216, the application of the electric pulse to the SMA element is terminated at 1218. Otherwise, the process is repeated at the next sample time.

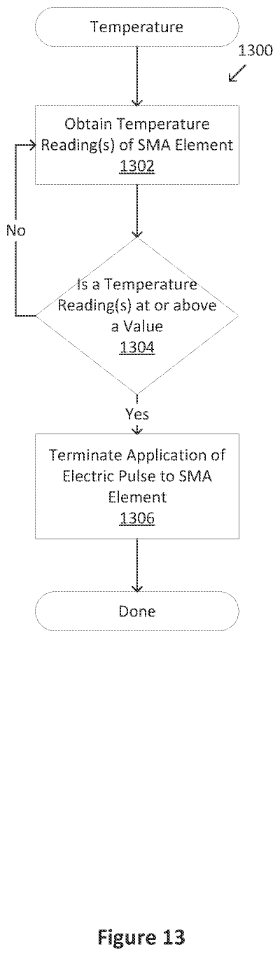

Another alternate factor that may be examined in deciding whether to trigger termination of the application of the electric pulse to the SMA element is temperature of the SMA element. As electricity is applied to the SMA element, the temperature of the SMA element rises. A signature temperature value or range may be associated with the inflection point where the actuation of the medicament pump 113 is completed. That signature temperature may be used as a trigger for the termination of the application of the electric pulse to the SMA element. FIG. 13 depicts a flowchart 1300 of illustrative steps that may be performed in exemplary embodiments to use temperature as a trigger. At 1302, one or more temperature readings of the SMA element are obtained from a temperature sensor. Multiple successive temperature readings may be obtained in some exemplary embodiments. At 1304, a check is made whether the one or more temperature readings are at or above a value indicative of the optimal termination point, which value may be based on a transition temperature of the SMA element and/or simple experimentation. The check may, for instance, compare an average of the last three temperature readings to the threshold value, may compare each of the temperature values to the threshold value, or may compare a single temperature value to the threshold. If the one or more temperature values (or an average thereof) are at or above the threshold, the application of the electric pulse to the SMA element is terminated at 1306. Otherwise, the steps are repeated at 1302.

In other exemplary embodiments, both time and temperature may be examined to determine whether to terminate application of the electric pulse to the SMA elements. At 1402, one or more temperature readings are obtained from a temperature sensor. At 1404, if the one or more temperature reading(s) are not greater than or equal to the threshold temperature, the steps are repeated for a next sample time at 1402. If the one or more temperature reading(s) are greater than or equal to the threshold, the time since application of the electric pulse is referenced. Specifically, at 1406, the time since application of the electric pulse to the SMA element is determined. If the time is greater than the minimum time as checked at 1408, the application of the electrical pulse to the SMA element is terminated at 1410. Otherwise, the steps are repeated for the next sample time at 1402.

In some exemplary embodiments, multiple factors may be examined to determine whether to terminate application of the electric pulse to an SMA element. Confidence intervals may be defined based on values for the factors. The decision to terminate may be based on whether the values for the factors fall within a specified confidence interval in some embodiments.

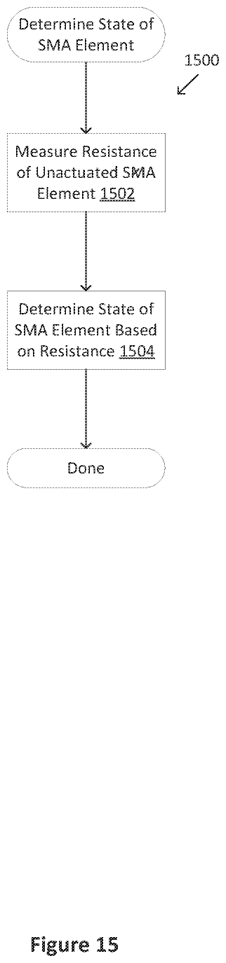

In some exemplary embodiments, the unactuated SMA element may be monitored to control when a pulse is initiated. FIG. 15 depicts a flowchart 1500 depicting illustrative steps that may be performed in exemplary embodiments to determine the state of an SMA element, such as an SMA wire. It is assumed that the SMA elements are in an opposing relationship such that they actuate against each other and that the SMA elements are alternately actuated. At 1502, the resistance of the unactuated SMA element is measured, such as by measuring the current and voltage and calculating resistance from those measured values.

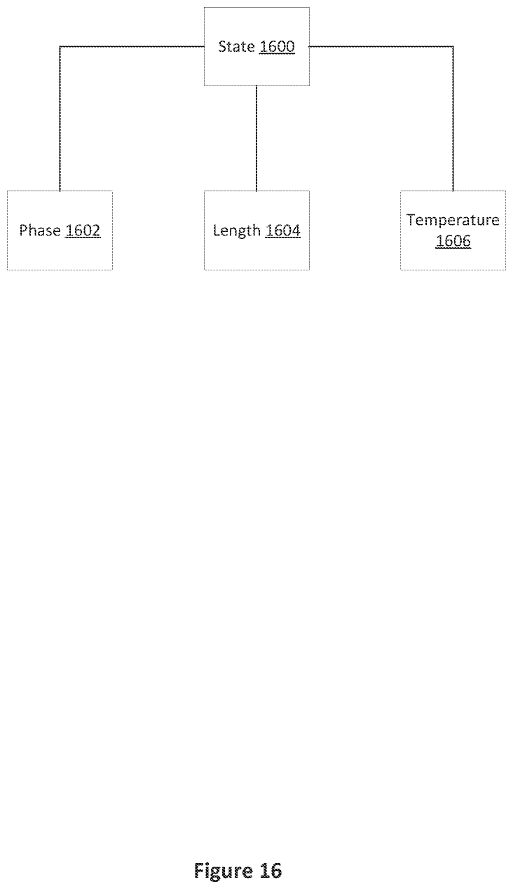

At 1504, the state of the unactuated SMA element may be determined from the resistance measurement. FIG. 16, depicts what sort of state information 1600 regarding the unactuated SMA element may be determined from the resistance. First, the state 1602 of the SMA element may be determined. For instance, the resistance may identify whether the SMA element is in an austenite phase, a martensite phase, or a mixture thereof. When the SMA element is in a mixed phase, the resistance may identify where the SMA element is in the transition between the phases. The resistance may also identify the length 1604 of the SMA element. The length 1604 is related to the phase because the SMA element shortens in the austenite phase and returns to its original length when it returns to the martensite phase. The temperature 1606 of the SMA element may also be determined from the resistance.

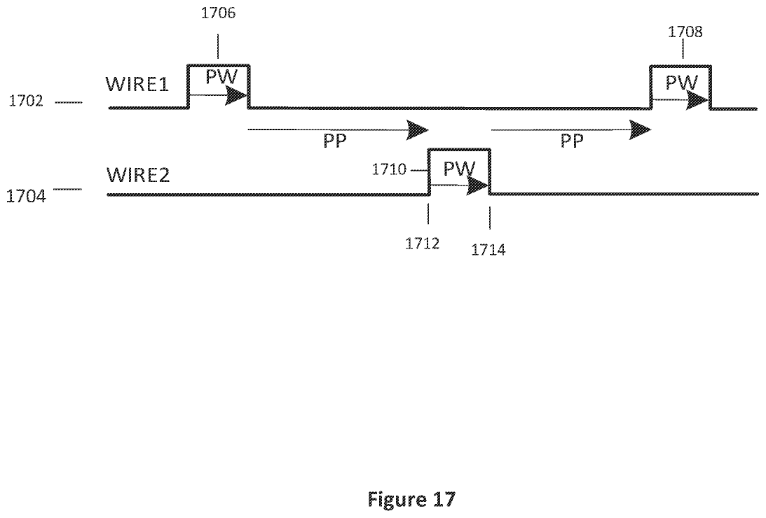

FIG. 17 depicts an illustrative depiction of the electrical pulses being applied to a first SMA wire 1702 and to a second SMA wire 1704. In the depiction, electrical pulses 1706 and 1708 are applied to SMA wire 1702. In between these electrical pulses 1706 and 1708, an electrical pulse 1710 may be applied to the other opposing SMA wire 1704. The pulse widths (PWs) of the electrical pulses 1706, 1708, and 1710 are shown. The pulse width represents the time between when an electrical pulse is initiated (e.g., 1712 for pulse 1710) and when the electrical pulse is terminated (1714). The pulse to pulse (PP) length is shown. The PP length (see the PP labelled arrows) represents the length of time between termination of an electrical pulse on one SMA wire and the initiation of an electrical pulse on the other SMA wire.

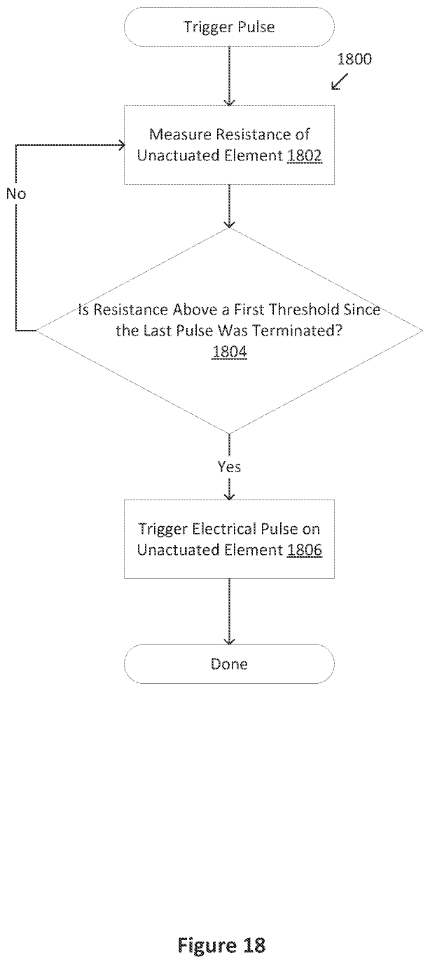

FIG. 18 depicts a flowchart 1800 of illustrative steps that may be performed in exemplary embodiments to decide when to trigger the initiation of an electrical pulse to an unactuated SMA element. The choice of when to trigger the initiation of the electrical pulses determines the PP timing. The aim of the choice of this timing in some exemplary embodiments is to minimize energy loss. Cooling an SMA element all the way down to ambient temperature is not efficient and results in wasted energy application. In these exemplary embodiments, the initiation of the application of an electrical pulse to the unactuated SMA element takes place when the unactuated SMA element has sufficiently cooled (or sufficiently transitioned to the martensite phase from the austenite phase, or sufficiently lengthened from its contracted length to return to its uncontracted or original length) but not all the way to ambient temperature. These embodiments may choose to apply the electrical pulse to the unactuated SMA element when the cooling is enough to stretch or de-twin the SMA element. Thus, the pulse widths are shorter and energy is saved. On the other hand, however, actuating before the SMA element has sufficiently relaxed will waste energy. This ideal window in which to initiate the application of an electrical pulse to the unactuated SMA element(s) will vary among SMA elements and may depend upon the materials (e.g., the alloys) that make up the SMA element, the length of the SMA element, the age of the SMA element, etc.

With reference again to FIG. 18, at 1802, the resistance of the unactuated SMA element is determined, such as has been described above, from voltage and current measurements, for example. The measuring of the resistance occurs after the last applied pulse to the other opposing SMA element is terminated. In some instances, a delay may be built in before resistance measurements are gathered. At 1804, the measured resistance is compared to a first threshold. The first threshold may be an empirically derived value that indicates that the unactuated SMA element (2002 in this case) has sufficiently cooled so that there is detwinning. If the measured resistance is above a threshold value (e.g., above a first threshold value since the last pulse was terminated), the process repeats at 1802. There may be a built-in delay between successive measurements at 1802. If the measured resistance is above the first threshold, it is an indication that there is sufficient cooling, and at 1806, the triggering of an electrical pulse occurs so that an electrical pulse is applied to the previously unactuated SMA element (i.e., 2004 or 1704 in this case).

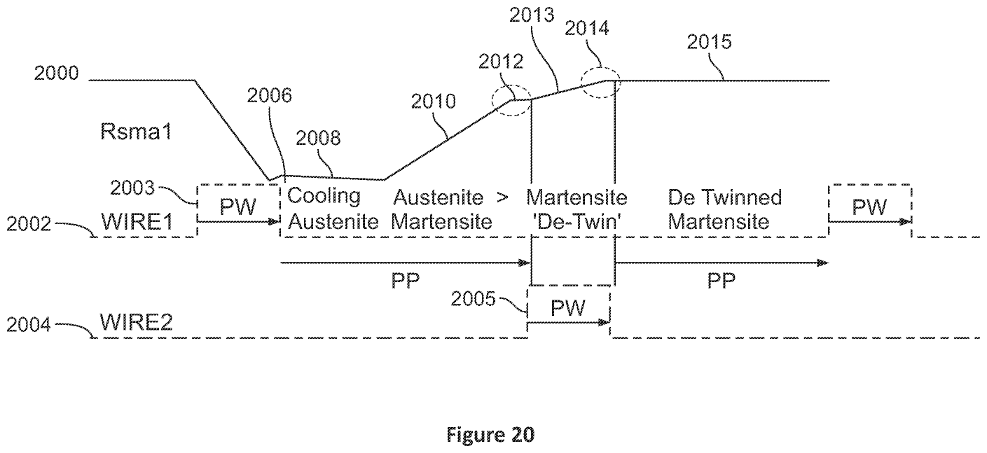

FIG. 20 depicts an example of the resistance values 2000 for the pulse pattern previously shown in FIG. 17. At the end of pulse 2006 for the SMA element 2002 (“wire 1”), the SMA element 2002 begins to cool because the electrical pulse is no longer applied. The resistance during this period 2008 stays changes only minimally. When the cooling is sufficient, the SMA element begins the transition back to the martensite phase, so the resistance begins to increase rapidly (i.e., more than at other periods) as can be seen at 2010. When the resistance surpasses the first threshold (see 2012), the electrical pulse is applied to the opposing SMA element 2004. The first threshold may be an empirically derived value that is known to represent a state as described above. In other embodiments, the threshold instead may be a rate of change in resistance or a derivative value over time.

The exemplary embodiments may also determine when to terminate an electrical pulse that is applied to an SMA element. In general, one wants to terminate the electrical pulse as soon as possible in order to save energy. The strategy of these exemplary embodiments described herein is to terminate the electrical pulse when the unactuated SMA element is still warm but no longer mostly austenite but before the phase transition to the martensite phase is complete.

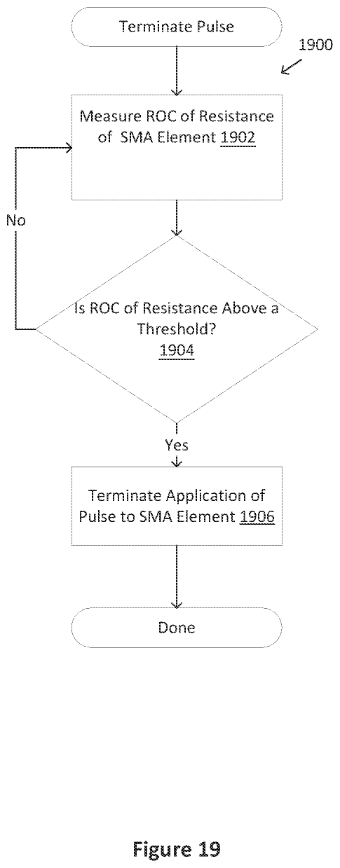

FIG. 19 depicts a flowchart 1900 of illustrative steps that may be performed in exemplary embodiments to decide when to terminate application of an electrical pulse to an SMA element based on the ROC of the resistance of the SMA element. The approach is like that described above in FIG. 6 for the single wire case. At 1902, the resistance of the SMA element is measured. At 1904, a check is made of whether the resistance is above a threshold. The threshold is an empirically derived value at which the electrical pulse should be terminated. If not, the process repeats beginning at 1902. In some instances, a delay may be added between 1904 and 1902. If so, the application of the electrical pulse to the SMA element is terminated at 1906. It should be appreciated that other approaches such as using the derivative (see FIG. 8) or the second derivative (see FIG. 9) may be used as well. Further, magnitude of resistance and ROC maybe used (see FIG. 10) or an approach like in FIG. 12 may be used.

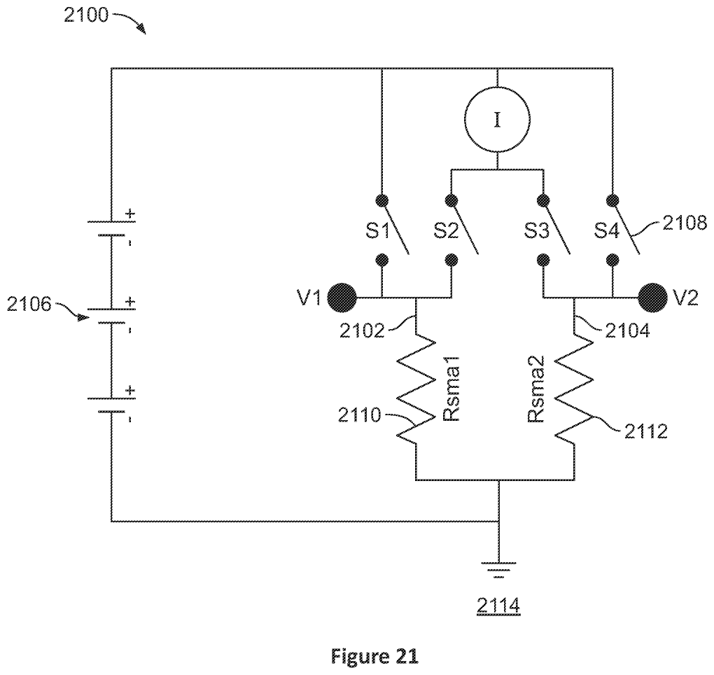

FIG. 21 depicts an illustrative electrical circuit 2100 of exemplary embodiments for measuring the resistance of the unactuated SMA elements (e.g., SMA2 2104), which in this case is an SMA wire arranged in an opposing configuration, like that described above, with another SMA wire (e.g., SMA1 2102). A power source 2106 provides power for the circuit 2100. Four switches 2108, designated as S1, S2, S3, and S4 are provided. The resistance values of the SMA wires are represented as resistors Rsma1 2110 and Rsma2 2112. The circuit is connected to ground 2114 as shown.

The application of a voltage to the SMA wires 2102 and 2104 causes current to flow through the SMA wires 2102 and 2104. Closure of switch S1 applies current to SMA1 2102, and closure of switch S4 applies power to SMA2 2104. Closure of switches S1 and S3 allows a current to be delivered to SMA1 2102 and SMA2 2104, respectively. The delivered current is set to a low level to conserve power and prevent the SMA wires 2102 and 2104 from being activated to undergo a phase change. The voltage of SMA1 2102 may be measured at V1, and the voltage of SMA2 2104 may be measured at V2. To measure the resistance of SMA2 2104 (the unactuated wire), the switch S3 may be closed so that current I flows through SMA2 2104. The resistance of SMA2 2104 may be calculated as V2/I.

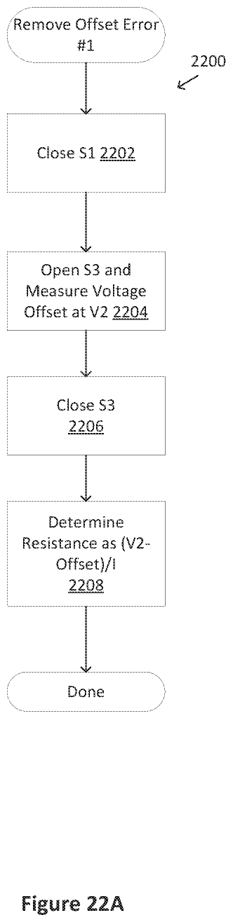

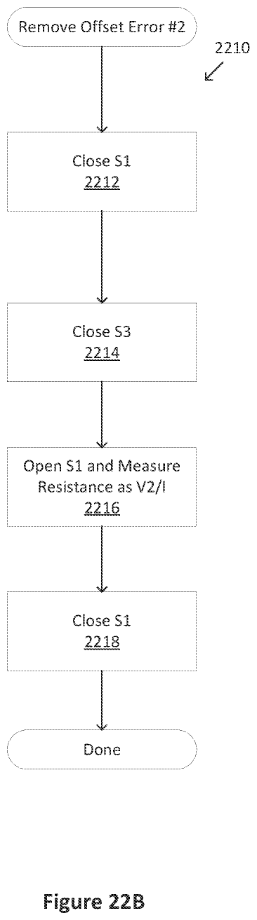

The voltage created by the large current of the driven SMA wire through the ground connection may create a voltage offset error due to ground impedance that may affect the calculated resistance of SMA2 2104. FIG. 22A depicts a flowchart 2200 of illustrative steps that may be performed in exemplary embodiments to account for the offset error. Initially, at 2202, switch S1 may be closed to drive SMA1 2102. At 2204, switch S3 may be opened, and the voltage offset may be measured at V2. At 2206, switch S3 is closed to direct current I to flow through SMA2 2104. At 2208, the resistance Rsma2 2112 may be calculated as (V2-voltage offset)/I.

The voltage offset alternatively may be taken into account as shown in the flowchart 2210 of FIG. 22B. At 2212, switch S1 is closed to cause current to flow through SMA1 2102. At 2214, switch S3 is closed to cause current I to flow through SMA2 2104. At 2216, switch S1 is opened to cause a brief pause in the driving of SMA1 2102, and the resistance of SMA2 2104 is calculated as the voltage at V2 divided by I. At 2218, switch S1 is closed again to drive SMA1 2102.

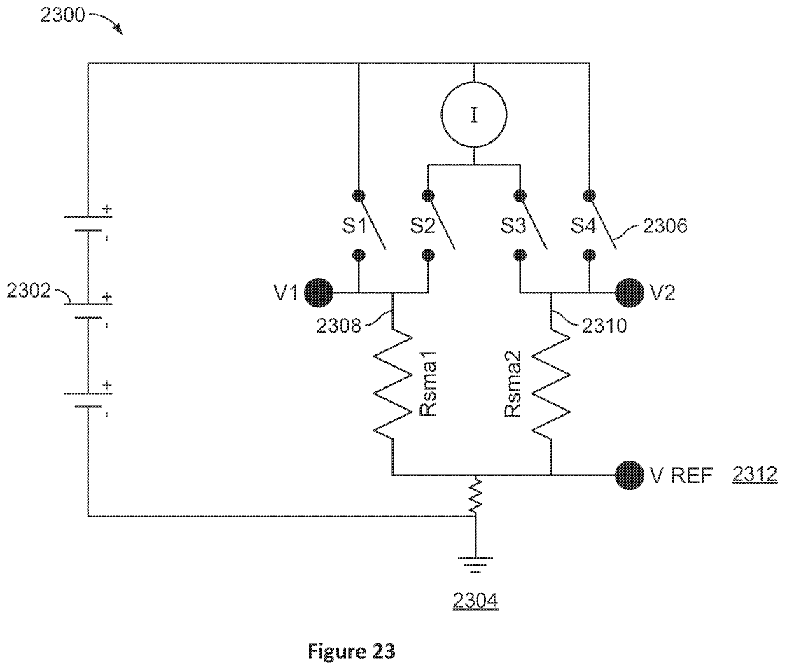

Another alternative to address the voltage offset problem is to add electrical connections to the electrical circuit as shown in electrical circuit 2300 of FIG. 23. The electrical circuit 2300 includes a voltage source 2302, a ground 2304, switches 2306 and SMA wires 2308 and 2310 as found in the electrical circuit 2100. However, a reference voltage VREF 2312 is used in a high impedance differential amplifier arrangement to remove the voltage offset while measuring V1 and V2.

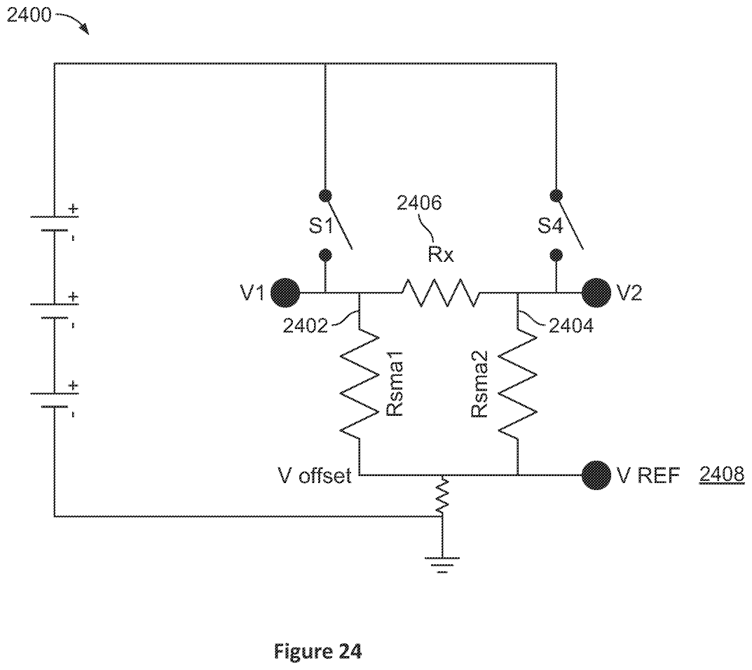

FIG. 24 depicts another alternative electrical circuit 2400 for measuring the resistance of SMA2 2404. The electrical circuit is similar to the electrical circuit 2300 shown in FIG. 23 with some notable differences. A large resistor 2406 crosses over between the SMA wires 2402 and 2406. The resistor 2406 provides a path for current to go through the SMA wire that is not driven (or that is not activated). This approach reduces the number of switches so that only two switches S1 and S2 are needed and also eliminates a current source. The VREF 2408 arrangement is like that depicted in FIG. 23.

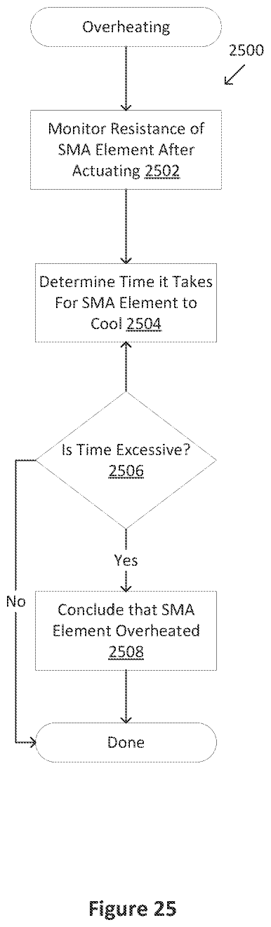

As was mentioned above, the resistance of the unactuated SMA element may be monitored to determine if the SMA element overheated by determining how long it takes for the SMA element to cool down as reflected in the resistance values. FIG. 25 depicts a flowchart 2500 of illustrative steps that may be performed in exemplary embodiments to identify overheating. At 2502, the resistance of the SMA element is monitored after it has had an electrical pulse applied to it. At 2504, based on the resistance, the temperature of the wire is determined, and the time it takes for the wire to cool to a specified temperature is determined. At 2506, a determination may be made to determine whether the time has been sufficient or whether the time was excessive. If the time was longer than anticipated given the ambient temperature, at 2508, a conclusion that the SMA element overheated may be reached. The cause of the overheating may be investigated and addressed.

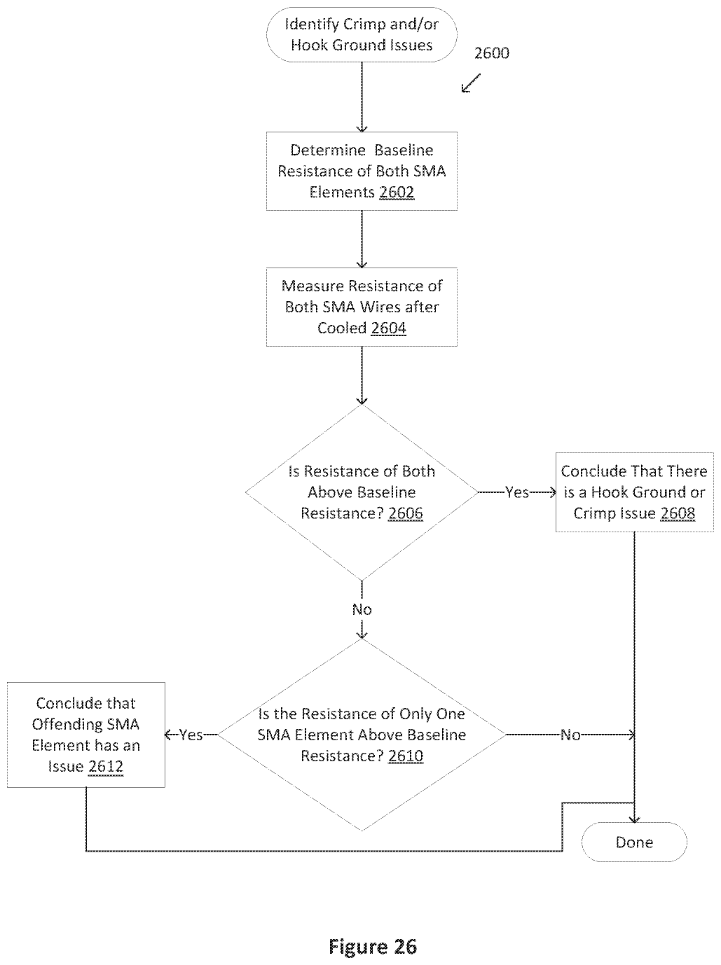

As was mentioned above, the monitoring of the resistance of the SMA elements may help identify problems with crimps, aging, lengthening, or other factors. FIG. 26 depicts a flowchart 2600 of illustrative steps that may be performed in exemplary embodiments to identify such problems. At 2602, the baseline resistance of both SMA elements may be determined. At 2604, the resistance of both SMA wires may be measured after the SMA elements have fully cooled. At 2606, a check is made to determine whether the resistance is above a baseline or a threshold resistance. If the resistance is above a baseline or a threshold resistance from both SMA elements, at 2608, it is concluded that there is an issue with the crimp or hook ground. If not, at 2610, a check is made to determine if one of the SMA elements has a resistance above the baseline or threshold resistance. If so, at 2612, it is concluded that the crimp for the offending SMA element with the elevated resistance has an issue or that another issue has arisen that significantly increased the circuit impedance. Otherwise, there is no issue identified.

While the exemplary embodiments have been described herein, it should be appreciated that various changes in form and detail may be made without departing from the scope of the appended claims.

1. A medicament delivery device for delivering medicament to a user, comprising:
a medicament reservoir for storing the medicament;
a medicament pump for pumping the medicament from the medicament reservoir for delivery to the user;
a shape memory alloy (SMA) element for causing actuation of the medicament pump to deliver the medicament;
a power source;
a processor configured to:
initiate application of an electric pulse from the power source to the SMA element to cause the medicament pump to deliver the medicament from the medicament reservoir;
determine resistance of the SMA element; and
based on the determined resistance of the SMA element over time, determine that the application of the electric pulse should be terminated; and
cause the application of the electric pulse to the SMA element to be terminated responsive to the determining that the application of the electric pulse should be terminated.

2. The medicament delivery device of claim 1, wherein the processor is configured to determine a rate of change of the resistance and compare the rate of change to a threshold in the determining that the application of the electric pulse should be terminated.

3. The medicament delivery device of claim 1, wherein the processor is further configured to determine a magnitude of change of the resistance of the SMA element since the initiating of the electric pulse, compare the determined magnitude of change of the resistance to a threshold, and cause the electric pulse to terminate in part based on the comparing to the threshold.

4. The medicament delivery device of claim 1, wherein the processor is configured to collect voltage readings and current readings for the SMA element over time and as part of the determining of the resistance of the SMA element over time, to calculate the resistance of the SMA over time from the collected voltage readings and current readings.

5. The medicament delivery device of claim 4, wherein the processor causes the application of the electric pulse to the SMA element to be terminated by determining moving average values of subsets of the resistance values, determining at least one derivative or approximation of the derivative of the determined moving average values, and comparing the at least one derivative or the approximation of the derivative of the determined moving average values to a threshold as part of the determining that the application of the electric pulse should be terminated.

6. The medicament delivery device of claim 1, wherein the SMA element is an SMA wire.

7. The medicament delivery device of claim 1, wherein the determining that the application of the electric pulse should be terminated comprises determining a second derivative or an approximation of a second derivative of one of the resistance values or averages of successive ones of the resistance values and based on the second derivative or the approximation of the second derivative, determining that the application of the electric pulse should be terminated.

8. A medicament delivery device, comprising:
a medicament reservoir for storing medicament;
a pump for pumping medicament from the medicament reservoir;
a shape memory alloy (SMA) element for actuating the pump;
a power source;
a processor configured to:
cause an electric pulse from the power source to be applied to the SMA element to cause actuation of the medicament pump to output medicament from the medicament reservoir;
monitor resistance values of the SMA element; and
terminate the application of the pulse to the SMA based on a metric reflective of the resistance values.

9. The medicament delivery device of claim 8, wherein the medicament delivery device is a wearable insulin pump and the medicament is insulin.

10. The medicament delivery device of claim 8, wherein the processor is configured to perform filtering of the resistance values.

11. The medicament delivery device of claim 8, wherein the power source is a battery or a capacitor.

12. The medicament delivery device of claim 8, wherein the SMA element is coupled to a component that drives actuation of the medicament pump.

13. The medicament delivery device of claim 8, wherein the SMA element shrinks in length responsive to application of the electric pulse.

14. The medicament delivery device of claim 8, wherein the metric reflective of the resistance values is a metric of a rate of change over time concerning the resistance values or of averages of the resistance values.

15. The medicament delivery device of claim 14, wherein the metric of a rate of change over time concerning the resistance values is a derivative of the resistance values or averages of the resistance values.

16. The medicament delivery device of claim 14, wherein metric of a rate of change over time concerning the resistance values is a second derivative of the resistance values or averages of the resistance values.

17. A medicament delivery device, comprising:
a medicament reservoir for storing medicament;
a pump for pumping medicament from the medicament reservoir;
a shape memory alloy (SMA) element for actuating the pump;
a power source;
a temperature sensor for sensing temperature of the SMA element;
a clock for outputting an indication of time; and
a processor configured to:
cause an electric pulse from the power source to be initially applied to the SMA element to actuate the medicament pump to output medicament from the medicament reservoir;
monitor temperature values of the SMA element that were measured by the temperature sensor; and
terminate the application of the pulse to the SMA element based on the temperature of the SMA element and time since the initial application of the electric pulse to the SMA element.

18. The medicament delivery device of claim 17, wherein the SMA element is a SMA wire.

19. The medicament delivery device of claim 17, wherein multiple successive temperature values must exceed a threshold before terminating the application of the pulse to the SMA element.

20. The medicament delivery device of claim 17, wherein the medicament delivery device is an insulin delivery device.
